(19) United States
(12) Patent Application Publication
(10) Pub. No.: US 2004/0180131 A1
(43) Pub. Date: Sep. 16, 2004

(54) Stent coating method

(75) Inventor: Peiwen Cheng, Santa Rosa, CA (US)

Correspondence Address: Michael J. Jaro, IP Legal, 3576 Unocal Place, Santa Rosa, CA, 95403, US

(73) Assignee: MEDTRONIC AVE.

(21) Appl. No.: 10/389,084

(22) Filed: Mar. 14, 2003

(51) Int. Cl. A61L 2/00

(52) U.S. Cl. 427/2.24

(57) ABSTRACT

Methods for coating a stent according to the invention include mixing a plurality of compounds to form a solution and applying the solution to a stent frame. The solution is dried on the stent frame to substantially evaporate solvent(s). In one embodiment, the solution includes at least one therapeutic agent, a poly(□-caprolactone) polymer, and a tetrahydrofurane solvent. In another embodiment, the solution includes a Resten-NG therapeutic agent, at least one polymer, and at least one solvent including methanol. In yet another embodiment, the solution includes a podophyllotoxin therapeutic agent, at least one poly(n-butylmethacrylate-co-vinylacetate) polymer, and at least one solvent.

### TECHNICAL FIELD OF THE INVENTION

The present invention relates generally to the field of implantable medical devices. More particularly, the invention relates to a method for coating a stent.

### BACKGROUND OF THE INVENTION

Balloon angioplasty has been used for the treatment of narrowed and occluded blood vessels. A frequent complication associated with the procedure is restenosis, or vessel re-narrowing. Within 3-6 months of angioplasty, restenosis occurs in almost 50 percent of patients. To reduce the incidence of re-narrowing, several strategies have been developed. Implantable devices, such as endovascular stents, have been used to reduce the rate of angioplasty related restenosis by about half. The use of such devices has greatly improved the prognosis of these patients. Nevertheless, restenosis remains a formidable problem associated with the treatment of narrowed blood vessels.

Stents are generally short flexible cylinders constructed of metal or various polymers that are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, U.S. Pat. No. 5,421,955 to Lau, and U.S. Pat. No. 5,935,162 to Dang. Stents may be self-expanding or expanded in sympathy with an inflatable balloon. The stents are typically compressed to a smaller profile prior to deployment.

The stent, or other prosthetic device, may be implanted during interventional procedures such as balloon angioplasty to reduce the incidence of vessel restenosis. To improve device effectiveness, the stent may be coated with one or more therapeutic agents providing a mode of localized drug delivery. The therapeutic agents may limit or prevent the restenosis. For example, antithrombogenic agents such as heparin or clotting cascade IIb/IIIa inhibitors (e.g., abciximab and eptifibatide) may be coated on the stent thereby diminishing thrombus formation. Such agents may effectively limit clot formation at or near the implanted device. Furthermore, antiangiogenesis agents, antiarteriosclerotic agents, antiarythmic agents, antibiotics, antidiabetic agents, antiendothelin agents, antinflammatory agents, antimitogenic factors, antioxidants, antiplatelet agents, antiproliferative agents, antisense agents, calcium channel blockers, clot dissolving enzymes, growth factor inhibitors, growth factors, immunosuppressants, nitrates, nitric oxide releasing agents, vasodilators, virus-mediated gene transfer agents, agents having a desirable therapeutic application, combinations of the above, and a variety of other drugs may also be included to modulate localized immune response, limit hyperplasia, or provide other benefits. Therapeutic agents provided as coatings on implantable medical devices may effectively limit restenosis and reduce the need for repeated revascularization treatments.

Several prior art strategies for providing a uniform stent coating involve dissolving a composition of drug or other therapeutic agent and (co)polymer in a common solvent. The liquid composition may be applied by dipping, spraying, or other methods. The liquid coating then dries to a solid coating upon the stent forming a drug/polymer reservoir in the dried film. The polymer acts as a matrix providing a framework for the drug while the solvent contributes greatly to the smoothness, morphology, and uniformity of the coating. The solvent should meet various criteria to provide an acceptable stent coating. For example, the solvent should have low toxicity, a reasonable evaporation rate, low residual after process, no interaction with drug or polymer, stable shelf-life, etc. A solvent pair or mixed solvent may be used when the drug and polymer do not dissolve in one solvent. For example, a hydrophobic drug with a hydrophilic polymer or a hydrophilic drug with a hydrophobic polymer may require dissolution in a mixed common solvent.

In the case controlled drug release polymeric coatings of medical devices, such as stents, the smoothness of the coating is an important factor in determining implantation result. In most common approaches to device coating, a solution of drug(s) and polymer(s) dissolved in solvent(s) is applied to the device surface, allowed to dry, and form a thin film. Depending on the solvent(s) chosen, the resulting coating morphology may be quite different. Good solvents generally will extend the polymer chain in the spray solution and provide uniformly distributed drug in the polymer matrix. Poor solvents, however, may coil the polymer chain and provide a relatively rough surface potentially leading to thrombosis, proliferation, and/or restenosis. The nature of the solvent may also influence the drug releasing profile. A poor solvent may provide a non-homogeneous distribution of the drug resulting in drug clustering or clumping. This, in turn, may lead to an unpredictable discharge of the drug from the matrix; a clinically undesirable condition. Therefore, it would be desirable to provide a strategy for coating a medical device, such as a stent, that provides a relatively smooth coating morphology.

Accordingly, it would be desirable to provide a strategy for coating a stent that would overcome the aforementioned and other disadvantages.

### SUMMARY OF THE INVENTION

One aspect according to the invention provides a method for coating a stent. The method includes mixing at least one therapeutic agent, a poly(ε-caprolactone) polymer, and a tetrahydrofurane solvent to form a solution. The solution is applied to a stent frame. The solution is dried on the stent frame to substantially evaporate the solvent. The therapeutic agent may include etoposide, sulindac, and/or tranilast. The solution may have a weight-to-weight ratio of therapeutic agent and polymer to solvent of about one percent. The solution may be applied by spray coating and/or dipping. A cap coating may be applied to the stent frame.

Another method for coating a stent according to the invention includes mixing a Resten-NG therapeutic agent, at least one polymer, and at least one solvent including methanol to form a solution. The solution is applied to a stent frame. The solution is dried on the stent frame to substantially evaporate the solvent. The polymer may include poly(□-caprolactone), poly(ethylene-co-vinylacetate), poly(hydroxy-alkyl-methacrylate),and/or poly(n-vinyl-pyrrolidone). The solvent may include chloroform and/or water. The solution may be applied by spray coating and/or dipping. A cap coating may be applied to the stent frame. Applying the cap coating may include mixing a poly(n-butyl-methacrylate-co-vinylacetate) polymer and an acetone solvent to form a cap solution. The cap solution may be applied to the coated stent frame and dried.

Another method for coating a stent according to the invention includes mixing a podophyllotoxin therapeutic agent, at least one poly(n-butyl-methacrylate-co-vinylacetate) polymer, and at least one solvent to form a solution. The solution is applied to a stent frame. The solution is dried on the stent frame to substantially evaporate the solvent. The solvent may include tetrahydrofurane and/or acetone. The solution may be applied by spray coating and/or dipping. A cap coating may be applied to the stent frame. Applying the cap coating may include mixing poly(n-butyl-methacrylate-co-vinylacetate) polymer and a cap solvent to form a cap solution. The cap solution may be applied to the coated stent frame and dried. The cap solvent may include tetrahydrofurane and/or acetone. A podophyllotoxin therapeutic agent may be included in the cap solution.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is a perspective view of an exemplary prior art stent compatible with the disclosed coating methods according to the present invention; FIG. 2 is a pictomicrograph that illustrates a stent portion that has been coated with a solution that includes a sulindac therapeutic agent, PCL polymer, and acetone solvent;

FIG. 3 is a pictomicrograph that illustrates a stent portion that has been coated with a solution in accordance with the present invention, the solution includes a sulindac therapeutic agent, PCL polymer, and THF solvent; FIG. 4 shows a percentage drug eluted over time from stents coated in accordance with the present invention, the stents with and without a cap coat;

FIG. 5 shows a percentage drug eluted over time from a stent including a base and cap coat in accordance with the present invention, the stent includes acetone used as the cap coat solvent; and

FIG. 6 shows a percentage drug eluted over time from a stent including a base and cap coat in accordance with the present invention, the stent cap coat includes THF used as the cap coat solvent.

### DESCRIPTION OF THE PRESENTLY PREFERRED EMBODIMENTS

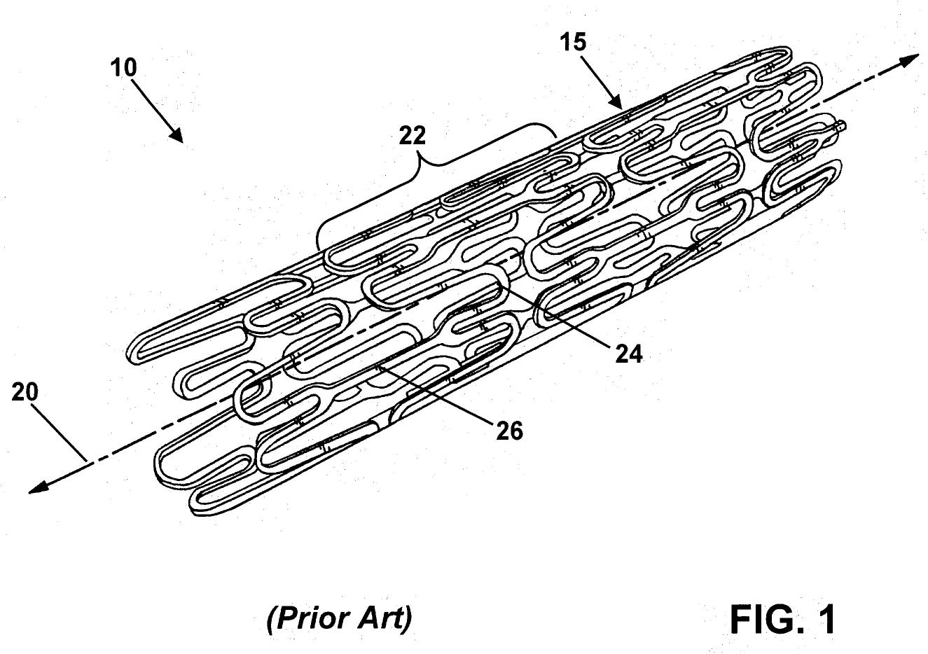

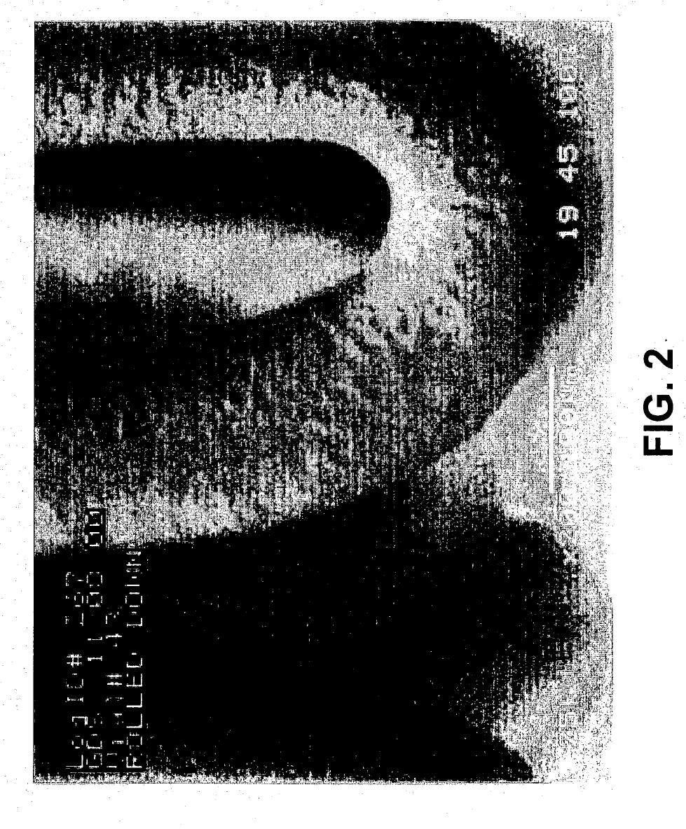

Referring to the drawings, wherein like reference numerals refer to like elements, FIG. 1 is a perspective view of a prior art stent 10 that may be compatible with coating methods according to the present invention. Those skilled in the art will recognize that numerous stents, grafts, and implantable prosthetic devices are compatible with the disclosed coating methods and that the described stent 10 is an illustration of merely one such device. The stent 10 is an example of a wire-tubular hybrid stent disclosed by U.S. Pat. No. 5,935,162 issued to Dang.

The stent 10 includes a generally tubular body defining a passageway extending along a longitudinal axis 20. The stent 10 includes a frame 15 formed from a plurality of cylindrical segments 22 arranged successively along the longitudinal axis 20. Each of cylindrical segments 22 has a length along the longitudinal axis 20 and includes a plurality of W-shaped elements 24. The W-shaped elements 24 open in alternating directions along the longitudinal axis 20 about the perimeter or circumference of the cylindrical segments 22. The W-shaped elements 24 are connected to each other by a tie member 26 that is attached to center sections of each of the W-shaped elements 24.

The stent 10 is shown in an expanded state in which the cylindrical segments 22 have been expanded radially outward from the longitudinal axis 20. The stent 10 may be compressed into a smaller diameter for delivery within a vessel lumen at which point the stent 10 may be expanded to provide support to the vessel. The stent 10 may be of the self-expanding variety and manufactured from nickel titanium alloys and other alloys that exhibit superlastic behavior (i.e., capable of significant distortion without plastic deformation). Alternatively, the stent 10 may be designed to be expanded by a balloon or some other device, and may be manufactured from an inert, biocompatible material with high corrosion resistance. The biocompatible material should ideally be plastically deformed at low-moderate stress levels. Suitable materials for the self-expanding or balloon-expandable stents include, but are not limited to, polymeric material, aluminum, glass, ceramic, tantalum, stainless steel, nitinol (a nickel titanium, thermo-memoried alloy material), titanium, nickel, niobium, high carat gold K 19-22, cobalt alloys, certain other alloys, and combinations thereof.

One or more coatings may be applied to the stent 10 using a method according to the invention. The coating may be formed from a solution including one or more therapeutic agents and polymers dissolved in one or more solvents. The therapeutic agents, or drug, used in the present invention may or may not be micronized. When the therapeutic agent is micronized, the agent particle size may be greater than about 10 □m. When the drug is micronized, the agent particle size is preferably 5-12 □m and more preferably about 5 □m. In some instances, multiple therapeutic agents may be included in one or more of the coating layers.

The polymer used for the coating may be biodegradable or non-biodegradable, and are necessarily biocompatible to avoid any deleterious effects. The polymer may be biodegradable or non-biodegradable depending on a desired rate of release or desired degree of polymer stability. A biodegradable polymer may be preferred since, unlike the non-biodegradable polymer, it will not remain long after implantation whereby it may cause an undesirable, chronic local response. Furthermore, a biodegradable polymer may not present a risk that over an extended period of time there could be an adhesion loss between the stent 10 and coating caused by mechanical stresses on the stent 10. The adhesion loss may result in the coating dislodging potentially posing a risk to the patient. In some instances, multiple polymers may be included in one or more of the coating layers

The solvent may be chosen such that there is a proper balance of viscosity, deposition level of the polymer, solubility of the therapeutic agent, wetting of the stent 10, coating morphology smoothness, and evaporation rate of the solvent to properly coat the stent 10. In one embodiment, the therapeutic agent and the polymer are both soluble in a single solvent. In another embodiment, the therapeutic agent and polymer are both soluble in a mixed solvent or solvent pair. Preferably, the solvent chosen minimizes the aggregation or agglomeration of particles into collections of particles that may clog stent 10 frame openings when applied. Although the solvent may be dried completely from the coating during processing, it is advantageous for the solvent to be non-toxic, non-carcinogenic, and environmentally benign. Mixed solvents systems may also be used to control viscosity and evaporation rate. It is important that the solvent not react with or inactivate the therapeutic agent or react with the coating polymer.

Several strategies for providing a relatively smooth stent coating morphology will now be described. The process may start by preparing a solution for application to a stent. The following examples (1-3) describe embodiments of the invention for preparing a solution including at least one therapeutic agent, a poly(□-caprolactone) polymer, and a tetrahydrofurane solvent for application to the stent frame. The therapeutic agent may include etoposide, sulindac, and/or tranilast. In addition, the solution may have a weight-to-weight ratio of therapeutic agent and polymer to solvent of about one percent. For example:

### EXAMPLE 1

To prepare a therapeutic agent (e.g., antiproliferative drug) and polymer solution including 50/50 (w/w) etoposide//PCL (poly(□-caprolactone)) in 1% (w/w) THF (tetrahydrofurane), fill a 100 ml volumetric flask with THF. Take 5 bottles of etoposide (˜100 mg per bottle) and mark label with # 1, 2, 3, 4, and 5. Pre-weigh the # 1, 2, 3, 4, and 5 and record the weight. Inside of a hood, take few ml THF from volumetric flask and add to the drug bottle, rinse inside of bottleneck first, then shake the bottle. Use a pipette to transfer drug-THF solution into a 200 ml small neck glass bottle (pre-clean the bottle with soap, water and rinse with THF three times). Repeat same rinse procedure for #2, 3, 4, and 5. Let # 1, 2, 3, 4, and 5 sit in hood for 5 minutes, then re-cap the bottles. Move #1, 2, 3, 4, and 5 out of the hood and measure their post-weight. Calculate the total amount of etoposide transferred (e.g., 0.4895 g). Weigh the same amount of PCL (e.g., 0.4890 g). Add PCL into a small neck glass bottle. Perform the final calculation to get a total volume of THF needed (e.g., 109 ml). Add an additional 9 ml THF to 100 ml already present. Shake the etoposide, PCL, and THF solution until the PCL is substantially dissolved.

### EXAMPLE 2

To prepare a therapeutic agent (e.g., anti-inflammatory drug) and biodegradable polymer solution including 30/70 (w/w) sulindac/PCL (poly(□-caprolactone)) in 1% (w/w) THF (tetrahydrofurane), use a funnel and pipette to transfer 100 ml THF into a volumetric flask. Pour about 98 ml of THF through the funnel into the volumetric flask first. Then, use the pipette to add THF up to the 100 ml mark. Weigh out sulindac (e.g., 0.2526 g) in a weight boat. Transfer it to a small neck glass bottle. Use THF from the 100 ml volumetric flask to rinse the weighing boat three times to make sure all the sulindac is transferred into the small neck bottle. Transfer all the remaining THF from volumetric flask into the small neck bottle and mix. Weigh out PCL (e.g., 0.5938 g) in a weight boat. Transfer PCL into the small neck bottle. Shake the sulindac, PCL, and THF solution until the PCL is substantially dissolved.

### EXAMPLE 3

To prepare a therapeutic agent (e.g., anti-inflammatory drug) and biodegradable polymer solution including 50/50 (w/w) tranilast/PCL (poly(□-caprolactone)) in 1% (w/w) THF (tetrahydrofurane), a protocol essentially similar to that described in example 1 above may be used (i.e., substituting tranilast for etoposide).

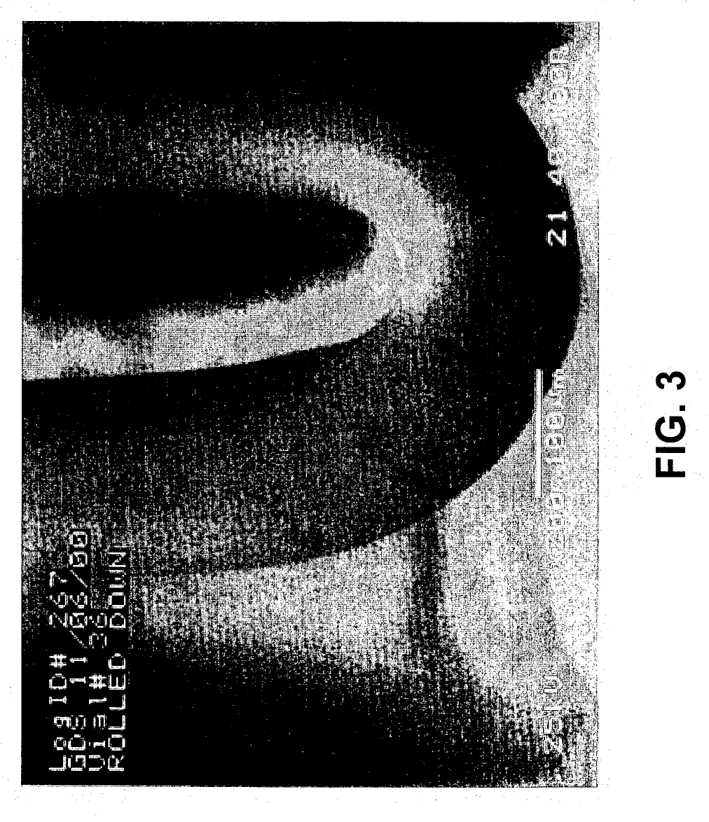

The use of solvent may influence morphology of the stent coating. For example, FIG. 2 shows a stent coated with a mixture including 20% sulindac and 80% PCL in acetone. FIG. 3 shows a stent coated with a mixture including 20% sulindac and 80% PCL in THF. Using THF as a solvent instead of acetone in this example provides a qualitative enhancement in smoothness and stent coating morphology. Therefore, the stent of FIG. 3 may provide superior drug delivery as well as other characteristics.

The solvent pair or mix solvent may be used when there is no suitable like solvent of the drug and polymer. For example, an antisense drug may be hydrophilic and, therefore, will not readily dissolve in a non-polar solvent such as chloroform or THF. Most polymers, however, are hydrophobic polymers and are not soluble in a polar solvent such as alcohol. Therefore, a pair of solvents may be used.

The following examples (4-6) describe embodiments of the invention for preparing a solution including a Resten-NG therapeutic agent, at least one polymer, and at least one solvent including methanol for application to the stent frame. The polymer may include poly(□-caprolactone), poly(ethylene-co-vinylacetate), poly(hydroxy-alkylmethacrylate), and/or poly(n-vinylpyrrolidone). The solvent may include chloroform and/or water. For example:

### EXAMPLE 4

To prepare a Resten-NG and biodegradable polymer solution including 50/50 (w/w) Resten-NG/PCL (poly(□-caprolactone)) in a mixed solvent including methanol, weigh out and then add Resten-NG (e.g., 0.1182 grams) and PCL (e.g., 0.1186 grams) into a small glass vial. Add 12.7 ml of chloroform into the vial. Shake the vial and a milky solution should form (e.g., an emulsion). Add 4.8 ml of methanol into the same vial and shake it well. The solution should become clear.

### EXAMPLE 5

To prepare Resten-NG and a multi-polymer solution including 33/67 (w/w) Resten-NG/PEVA (poly(ethylene-co-vinylacetate) and PHEMA ((poly(2-hydroxy-ethylmethacrylate)) in a mixed solvent including methanol, weigh out and then add Resten-NG (e.g., 0.052 grams) and PEVA (e.g., 0.098 grams) into a first small glass vial. Add 12.7 ml of chloroform into the first vial. Shake the vial and a milky solution should form (e.g., an emulsion). Weigh out and add PHEMA (e.g., 0.182 grams) into a second small glass vial. Add 4.8 ml of methanol into the second vial and shake well until PHEMA dissolves. Combine these contents of the first and second flasks. The solution should become clear.

### EXAMPLE 6

To prepare Resten-NG and polymer solution including 50/50 (w/w) Resten-NG/PVPP (poly(n-vinylpyrrolidone)) in a mixed solvent including methanol, weigh out and add Resten-NG (0.0964 g) and PVPP (e.g., 0.1022 grams) into a small glass vial. Add 23.6 ml of methanol and 1.0 ml of water into the vial. Shake the vial until the Resten-NG and PVPP are substantially dissolved.

The following examples (7-8) describe embodiments of the invention for preparing a solution including a podophyllotoxin therapeutic agent, at least one poly(n-butylmethacrylate-co-vinylacetate) polymer, and at least one solvent for application to the stent frame. The solvent may include tetrahydrofurane and/or acetone. For example:

### EXAMPLE 7

To prepare a podophyllotoxin therapeutic agent (e.g., anti-mitotic agent) and polymer solution including 50/50 (w/w) podophyllotoxin/Cyclops-2 (poly(n-butylmethacrylate-co-vinylacetate)) in THF (tetrahydrofurane), weigh out and add podophyllotoxin (e.g., 0.2952 g grams) and Cyclops-2 (e.g., 0.2954 grams) into a small glass vial. Add 65.8 ml of THF into the vial. Shake the podophyllotoxin, Cyclops-12, and THF solution until the PCL is substantially dissolved.

### EXAMPLE 8

To prepare a podophyllotoxin therapeutic agent (e.g., anti-mitotic agent) and polymer solution including 50/50 (w/w) podophyllotoxin/Cyclops-12 (poly(n-butylmethacrylate-co-vinylacetate)) in THF (tetrahydrofurane) or acetone, a protocol essentially similar to that described in example 7 above may be used (i.e., substituting Cyclops-12 for Cyclops-2, and (optionally) acetone for THF).

Once the solution has been prepared, it may be applied to the stent frame. The solution may then be dried to substantially evaporate the solvent. The solution may be applied by numerous strategies including painting, spraying, dipping, wiping, electrostatic deposition, vapor deposition, epitaxial growth, combinations thereof, and other methods known to those of ordinary skill in the art. It should be recognized that numerous coating configurations, such as partial and multiple coating layers, are possible. Furthermore, the coating topography and position may vary. For example, the coating may be formed on the stent inside, outside, or both areas. The coating may be formed of multiple layers of material to provide different therapies as the individual layers become depleted or as different layers biodegrade. Different coatings may be applied on the inside and the outside of the stent to provide different therapies on the lumen side and the tissue side of the stent. For ease of manufacture, the coating may be applied only on the outside of the stent to allow the stent to be held in place by a mandrel inside of the stent while the coating is applied. Examples of stent coating strategies are disclosed by U.S. Pat. Nos. 5,891,507 and 5,895,407 both to Jayaraman, which are incorporated by reference herein.

After one or more coatings have been applied to the stent and dried, a cap coating may be applied. The cap coating may be applied to the stent frame in a manner similar to that for the base coating(s). The cap coating is typically intended to reduce loss and/or damage of underlying base coating(s) as the stent is advanced through a tortuous vessel network to reach the implantation site. In addition, the cap coat may prevent portions of the base coating(s) from breaking off and resulting in embolization. Loss or damage of the base coating(s) may result in uncertainty in the delivered drug dosage. Additional drug loading of sometimes expensive therapeutic agents may then be required to achieve an effective drug dosage delivery.

The following examples (9-10) describe embodiments of the invention for preparing a cap solution for application to the already coated stent frame.

### EXAMPLE 9

To prepare a cap solution including a Cyclops-9 poly(n-butylmethacrylate-co-vinylacetate) polymer and acetone solvent, weigh out and add-Cyclops-9 (e.g., 0.2243 grams) in a small glass vial. Add 28.1 ml of acetone into the vial. Shake the vial until the Cyclops-9 is substantially dissolved. Alternatively, a THF (tetrahydrofurane) solvent may be substituted for acetone in a 90/10 (w/w) Cyclops-9/THF ratio.

### EXAMPLE 10

To prepare a cap solution including 25/75 (w/w) podophyllotoxin therapeutic agent, Cyclops-2 poly(n-butylmethacrylate-co-vinylacetate) polymer, and THF (tetrahydrofurane) solvent, weigh out and add podophyllotoxin (e.g., 0.2109 grams) and Cyclops-2 (0.6345 g) into a glass bottle. Add 89 ml of THF into the bottle. Shake the bottle until the podophyllotoxin and Cyclops-2 are substantially dissolved.

The cap coating examples may be compatible with a variety of base coats of the present invention. In one embodiment, the cap coat provided by example 9 (e.g., Cyclops-9 in acetone) may be used with a base coat provided by example 6 (e.g., Resten-NG, PVPP, in methanol-water mix). The use of a cap coat may influence the release profile of the therapeutic agent, or drug. For example, FIG. 4 shows percent drug eluted over time from a coated stent with and without a cap coat. The stent is base coated with a Resten-NG, PVPP in methanol/water mixed solvent and (optionally) cap coated with a Cyclops-9 in acetone mixture. The results demonstrate that adding a cap coat slows drug elution rate.

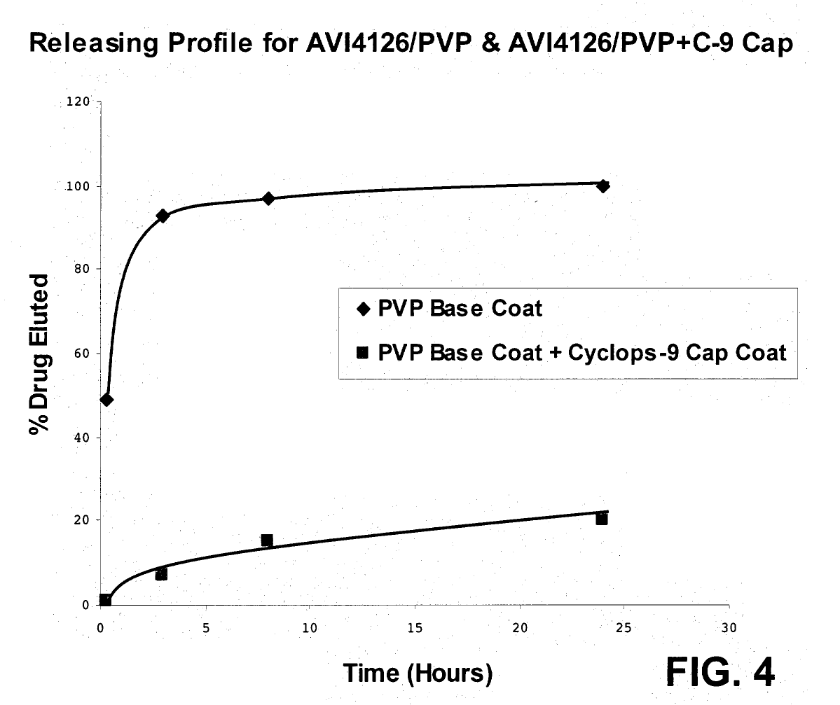

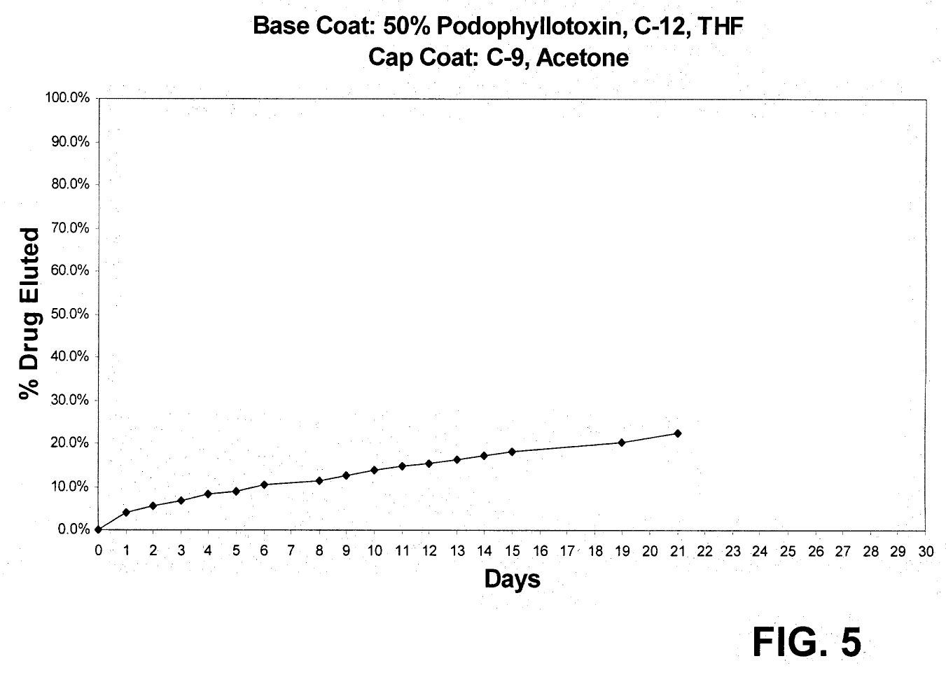

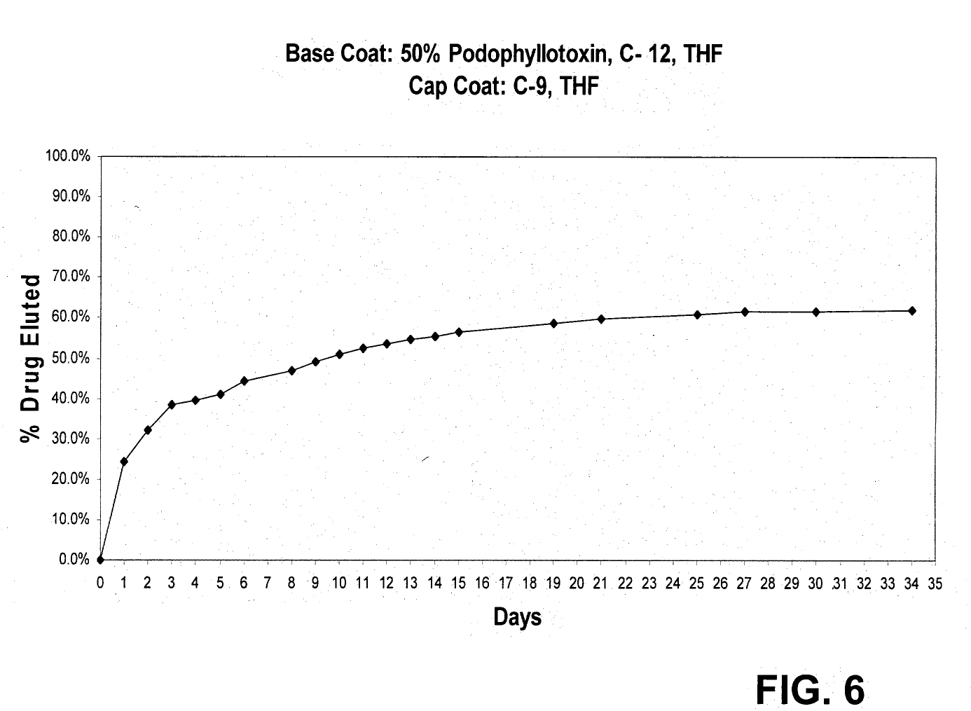

In another embodiment, the cap coat provided by example 9 (e.g., Cyclops-9 in acetone or THF) may be used with a base coat provided by example 8 (e.g., podophyllotoxin, Cyclops-12, in THF). Depending on the solvent chosen for the cap coat (e.g., acetone or THF), a different drug release profile may be obtained. For example, FIG. 5 shows percent drug eluted over time from a coated stent when acetone is used as the cap coat solvent. FIG. 6 shows the results from a like experiment using THF as the cap coat solvent. A marked increase in elution rate is measured when THF is used as the cap coat solvent. Thus, the drug release profile may be altered by using a different cap coat solvent.

In yet another embodiment, the cap coat provided by example 10 (e.g., podophyllotoxin in Cyclops-2 in THF) may be used with a base coat provided by example 7 (e.g., podophyllotoxin and Cyclops-2 in THF). Those skilled in the art will recognize that numerous cap coats may be used with the base coatings provided by the invention and that the above examples illustrate merely a portion of possible combinations.

It is important to note that the ratios of the solution components may be varied from the described examples while still providing a stent coating with favorable characteristics (e.g., smooth morphology). In addition, in certain instances, one or more of the components may be substituted, and one or more additional components (e.g., therapeutic agent, polymer, and/or solvent) may be added to the solution. The previous examples illustrate specific examples of solution preparation according to the invention, and are not intended to be comprehensive of all possible methodologies.

Suitable therapeutic agents that may be used with the methods according to the invention include, but are not limited to antiangiogenesis agents, antiarteriosclerotic agents, antiarythmic agents, antibiotics, antidiabetic agents, antiendothelin agents, antinflammatory agents, antimitogenic factors, antioxidants, antiplatelet agents, antiproliferative agents, antisense agents, antithrombogenic agents, calcium channel blockers, clot dissolving enzymes, growth factor inhibitors, growth factors, immunosuppressants, nitrates, nitric oxide releasing agents, vasodilators, virus-mediated gene transfer agents, agents having a desirable therapeutic application, combinations of the above, and the like. Specific examples of therapeutic agents include abciximab, angiopeptin, colchicine, eptifibatide, heparin, hirudin, lovastatin, methotrexate, streptokinase, taxol, ticlopidine, tissue plasminogen activator, trapidil, urokinase, and growth factors VEGF, TGF-beta, IGF, PDGF, and FGF.

Suitable biodegradable polymers that may be used include, but are not limited to polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like. Suitable other non-biodegradable polymers that may be used may be divided into at least two classes. The first class includes hydrophobic polymers such as polyolefins, acrylate polymers, vinyl polymers, styrene polymers, polyurethanes, polyesters, epoxy, nature polymers, their copolymers, blends, and copolymer blends, combinations thereof, and the like. The second class includes hydrophilic polymers, or hydrogels, such as polyacrylic acid, polyvinyl alcohol, poly(N-vinylpyrrolidone), poly(hydroxy-alkylmethacrylate), polyethylene oxide, their copolymers, blends and copolymer blends, combinations of the above, and the like.

Suitable solvents that may be used include, but are not limited to, ethyl acetate, N-methylpyrrolidone (NMP), and the like.

While the embodiments of the invention disclosed herein are presently considered to be preferred, various changes and modifications can be made without departing from the spirit and scope of the invention. For example, the stent configuration and method of coating the same are not limited to any particular design or sequence. Specifically, the stent frame, constitution, geometry, and size, and the method of applying the coating, specific ratios of the mixture components, layering and configurations of the coat, and method of drying may vary without limiting the utility of the invention. Furthermore, the ratios of the components used to may be varied to provide a viable mixture. Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. A method for coating a stent, comprising:
mixing at least one therapeutic agent, a poly(□-caprolactone) polymer, and a tetrahydrofurane solvent to form a solution;
applying the solution to a stent frame; and
drying the solution on the stent frame to substantially evaporate the solvent.

2. The method of claim 1 wherein the therapeutic agent is selected from a group consisting of etoposide, sulindac, and tranilast.

3. The method of claim 1 wherein the solution has a weight-to-weight ratio of therapeutic agent and polymer to solvent of about one percent.

4. The method of claim 1 wherein the solution is applied by spray coating.

5. The method of claim 1 wherein the solution is applied by dipping.

6. The method of claim 1 further comprising applying a cap coating to the stent frame.

7. A method for coating a stent, comprising:
mixing a Resten-NG therapeutic agent, at least one polymer, and at least one solvent including methanol to form a solution;
applying the solution to a stent frame; and
drying the solution on the stent frame to substantially evaporate the solvent.

8. The method of claim 7 wherein the polymer is selected from a group consisting of poly(□-caprolactone), poly(ethylene-co-vinylacetate), poly(hydroxyl-alkylmethacrylate), and poly(n-vinylpyrrolidone).

9. The method of claim 7 wherein the solvent is selected from a group consisting of chloroform and water.

10. The method of claim 7 wherein the solution is applied by spray coating.

11. The method of claim 7 wherein the solution is applied by dipping.

12. The method of claim 7 further comprising applying a cap coating to the stent frame.

13. The method of claim 12 wherein applying the cap coating comprises:
mixing a poly(n-butylmethacrylate-co-vinylacetate) polymer and a acetone solvent to form a cap solution;
applying the cap solution to the coated stent frame; and
drying the cap solution on the coated stent frame to substantially evaporate the acetone solvent.

14. A method for coating a stent, comprising:
mixing a podophyllotoxin therapeutic agent, at least one poly(n-butylmethacrylate-co-vinylacetate) polymer, and at least one solvent to form a solution;
applying the solution to a stent frame; and
drying the solution on the stent frame to substantially evaporate the solvent.

15. The method of claim 14 wherein the solvent is selected from a group consisting of tetrahydrofurane and acetone.

16. The method of claim 14 wherein the solution is applied by spray coating.

17. The method of claim 14 wherein the solution is applied by dipping.

18. The method of claim 14 further comprising applying a cap coating to the stent frame.

19. The method of claim 18 wherein applying the cap coating comprises:
mixing poly(n-butylmethacrylate-co-vinylacetate) polymer and a cap solvent to form a cap solution;
applying the cap solution to the coated stent frame; and
drying the cap solution on the coated stent frame to substantially evaporate the cap solvent.

20. The method of claim 19 wherein the cap solvent is selected from a group consisting of tetrahydrofurane and acetone.

21. The method of claim 19 wherein the cap solution includes a podophyllotoxin therapeutic agent.
